(19) United States
(12) Patent Application Publication
(10) Pub. No.: US 2008/0113030 A1
(43) Pub. Date: May 15, 2008

(54) Sustained release tamsulosin formulations

(76) Inventors: Ching-Fen Hsiao, Chiayi (TW); Sung-Jen Chen, Sinying City (TW); Ya-Ching Chang Chien, Kaohsiung (TW)

Correspondence Address: Frenkel & Associates, P.C., Suite 330, 3975 University Drive, Fairfax, VA, 22030, US

(21) Appl. No.: 11/595,620

(22) Filed: Nov. 9, 2006

(51) Int. Cl. A61K 31/18; A61K 9/16

(52) U.S. Cl. 424/490; 514/602; 514/603

(57) ABSTRACT

A sustained release tamsulosin formulation contains tamsulosin, a hydrophobic polymer, a microsphere forming agent and a diluent. The hydrophobic polymers include pH-dependent and pH-independent polymers are used as the release-modulating agent to control the dissolution profile of tamsulosin formulation so that the formulation releases tamsulosin slowly and continuously as the formulation passed through the stomach and gastrointestinal tract. The present invention further relates to a method for preparing the sustained release tamsulosin formulation.

### BACKGROUND OF THE INVENTION

1. Field of Invention

The present invention relates to a sustained release tamsulosin formulation.

2. Description of the Related Art

Tamsulosin is used in the treatment of benign prostatic hyperplasia (BPH), a condition characterized by enlargement of prostatic tissue, which results in obstruction of proximal urethra.

The mechanism of physiological action of tamsulosin HCl is through blocking the α-receptors actions in the cells of the urethra and prostate, so that the stress of a prostate is reduced and the difficulties with the flow of urine due to hypertrophy of the prostate are alleviated.

Tamsulosin is the common name for 5-[2-[[2-(2-ethoxyphenoxy)ethyl]amino]propyl]-2-methoxy-benzenesulfonamide. It was disclosed in EP 34432 and U.S. Pat. No. 4,731,478 as a pharmaceutically active substance having alpha-adrenergic blocking activity that is useful for treatment of cardiac insufficiencies and benign prostatic hyperplasia.

U.S. Pat. No. 4,772,475 discloses controlled-release pharmaceutical dosage forms comprising multiple granulate units containing tamsulosin HCl, microcrystalline cellulose and a release control agent. The release control agent includes an acrylic acid polymer, an acrylic acid copolymer or a cellulose derivative. More than 50% (w/w) microcrystalline cellulose was added into an oral sustained release formulation as a release-modulating agent. The granule gradually releases tamsulosin HCl from the granulated matrix. The patent suggests that an enteric coating is not needed. However, the high concentration of microcrystalline cellulose would increase the friction when a formulation mixture is kneaded and also elevate the temperature of the formulation mixture during the process of granulation. Also, acrylic acid polymer becomes glue-like under a high temperature, and the glue-like status of acrylic acid polymers in granulation procedures is unfavorable.

The conventional process for producing the granulate units comprises granulating a mixture of tamsulosin HCl, an unit-forming inert material such as microcrystalline cellulose and a release controlling agent comprising water and/or an aqueous emulsion, suspension or gel of a water-insoluble macromolecular substance or a solution of said macromolecular substance in an aqueous organic solvent. The macromolecular substance is preferably selected from a range of acrylic polymers. The release controlling agent serves essentially also as a binder in the granulation process. The resultant granulates may be used for making final dosage forms, capsules as well as tablets.

Example 1 of U.S. Pat. No. 4,772,475 illustrated the process. After sufficiently mixing 5 g tamsulosin HCl and 470 g microcrystalline cellulose, a mixture of 83.3 g (25 g as solid component) of Eudragit L 30 D-55 and 500 g of water was added thereto and the resultant mixture was granulated by a high-speed mixer. The granules obtained were spheres having particle sizes of 0.1 to 1.5 mm, mainly 0.2 to 1.0 mm in diameter.

U.S. Pat. No. 4,772,475 also disclosed that pellets of various compositions were prepared and tested for release characteristics according to standardized Pharmacopoeial method (paddle, 150 rpm). The reported results show that in one hour in simulated gastric fluid the release ranged from 16.2 to 60.4% of the active compound. Tablets made from some of the produced pellets, having 50.3 and 57.6% release, respectively, were also tested on human volunteers in comparison with conventional tablets and concentration of the active substance in blood plasma was measured. Peak plasma levels were reached 3 hours after ingestion (in comparison with 2 hours at conventional tablets), the total amount of tamsulosin in plasma being about 75% of that of the conventional tablet.

However, such release rate is generally not sufficiently for an extended-release dosage form. It would be desirable to provide an alternative, coated tamsulosin pellet having good release characteristics. There is still a need in the related art to provide a sustained release of tamsulosin formulation, which can overcome the problems of the glue-like status of acrylic acid polymer in the process of granulation, and maintain the desired extended-release effect.

### SUMMARY OF THE INVENTION

An aspect of the present invention is to provide a sustained release tamsulosin formulation, which comprises tamsulosin HCl or a pharmaceutically acceptable salt thereof, a hydrophobic polymer present at about 10% to about 50% w/w of the formulation, a microsphere forming agent present at about 10% to about 30% w/w of the formulation, and a diluent present at about 30% to about 50% w/w of the formulation. The formulation according to the present invention may be capable of releasing less than 5% of tamsulosin HCl or the pharmaceutically acceptable salt thereof during the first two hours.

Preferably, the diluent is selected from the group consisting of lactose, starch, mannitol, sodium hydroxylpropyl cellulose, sodium starch, microcrystalline cellulose, glyceryl behenate, talcum powder, stearic acid, stearic salt and sodium stearyl fumarate.

Preferably, the hydrophobic polymer is pH-dependent polymers or pH-independent polymers.

Preferably, the hydrophobic polymer is selected from the group consisting of methacrylic acid copolymer, sodium carboxymethyl cellulose, cellulose acetate, ethyl cellulose (EC), hydroxypropyl methyl-cellulose acetate succinate (HPMCAS) and cellulose acetate phthalate (CAP).

Preferably, the microsphere forming agent is glyceryl triacetate, glyceryl monostearate, glyceryl behenate, paraffin wax or carnauba wax. Preferably, tamsulosin HCl or the pharmaceutically acceptable salt thereof is present in the range of about 0.01% to about 3% w/w of the formulation.

More preferably, tamsulosin HCl or the pharmaceutically acceptable salt thereof is present in the range of about 0.03% to about 3% w/w of the formulation.

Another aspect of the present invention is to provide a method for preparing the sustained release tamsulosin formulation, which comprises preparing a mixture by mixing water-soluble tamsulosin HCl or the pharmaceutically acceptable salt thereof, a microsphere forming agent, a release modulating agent and a diluent, preparing a film coating premix, forming the mixture into granules, and coating the film coating premix on the granules.

Preferably, the film coat premix is dissolved in a solvent selected from the group consisting of water and organic solvents.

Preferably, the hydrophobic polymer is selected from the group consisting of methacrylic acid copolymer, sodium carboxymethyl cellulose, cellulose acetate, ethyl cellulose (EC), hydroxypropyl methyl-cellulose acetate succinate (HPMCAS) and cellulose acetate phthalate (CAP).

Preferably, the microsphere forming agent is glyceryl triacetate, glyceryl monostearate, glyceryl behenate, paraffin wax or carnauba wax.

Preferably, the film coat premix is prepared using more than one of ethylcellulose, triethyl citrate, methacrylic acid copolymer and talcum powder.

Other aspects, advantages and novel features of the invention will become more apparent from the following detailed description when taken in conjunction with the accompanying drawings.

### BRIEF DESCRIPTION OF THE DRAWINGS

FIG. 1 shows the tamsulosin HCl plasma concentrations under empty-stomach (fasted) and well-fed (fed) conditions after orally administering the standard sustained release tamsulosin formulation obtained from Example 1 of the present invention as capsules to humans.

FIG. 2 shows the tamsulosin HCl plasma concentrations under empty-stomach (fasted) and well-fed (fed) conditions after orally administering the standard sustained release tamsulosin formulation obtained from Example 2 of the present invention as capsules to humans; and

FIG. 3 shows the tamsulosin HCl plasma concentrations under empty-stomach (fasted) and well-fed (fed) conditions after orally administering a commercial sustained release tamsulosin formulation of the prior art as capsules.

### DETAILED DESCRIPTION OF THE INVENTION

An aspect of the present invention is to provide a sustained release tamsulosin formulation, which comprises tamsulosin HCl or a pharmaceutically acceptable salt thereof, a hydrophobic polymer present at about 10% to about 50% w/w of the formulation, a microsphere forming agent present at about 10% to about 30% w/w of the formulation, and a diluent present at about 30% to about 50% w/w of the formulation, wherein formulation releases less than 5% of tamsulosin HCl or the pharmaceutically acceptable salt thereof during the first two hours.

The term “sustained release tamsulosin formulation” as used herein refers to a formulation or dosage unit according to this invention that, after administered to a human, is capable of being slowly and continuously dissolved and absorbed in the gastrointestinal tract over a period of time.

The term “microsphere forming agent” as used herein refers to a binder according to the present invention for use in granule formation. The microsphere forming agent according to the present invention may also be employed for use in making the size of the formed granules equalized.

The term “fluctuation” as used herein refers to the change of the concentration of tamsulosin HCl in a human body. A smaller fluctuation means a more stable of the concentration of tamsulosin in a human body, e.g., in blood plasma.

Another aspect of the present invention is to provide a method for preparing the sustained release tamsulosin formulation, which comprises preparing a mixture by mixing water-soluble tamsulosin, a microsphere forming agent, a release modulating agent and a diluent, preparing a film coating premix, forming the mixture into granules, and coating the film coating premix on the granules.

According to the present invention, an effective, modified sustained release tamsulosin formulation can be prepared so that the formulation exhibits a controlled dissolution release profile. In preferred embodiments, when the dissolution release profile of the sustained release tamsulosin formulation was measured, less than 1 ng/ml of tamsulosin HCl was released during the first two hours in simulated gastric fluid in basket apparatus at 100 rpm. Accordingly, once the sustained release tamsulosin formulation of the present invention was ingested by a patient, tamsulosin HCl was released into his body at a rate that is characterized by minimizing the release during the sustained release tamsulosin formulation residence time in the stomach. More advantageously, in preferred embodiments according to the present invention, the sustained release tamsulosin formulation is prepared as granules. The granule size, the materials and amount of the coating can be decided such that the resultant collection of coated granules exhibits at least one of the following release profiles in simulated intestinal fluid (e.g. phosphate buffer of pH 6.8 as sometimes referred to herein): about 1 ng/ml of tamsulosin HCl released in 2 hours, about 3 ng/ml of tamsulosin HCl released in 4 hours, and about 4 ng/ml of tamsulosin HCl released in 6 hours.

More advantageously, according to the present invention, the sustained release tamsulosin formulation shows stable release rate in both well-fed and empty-stomach conditions.

Polymers have been widely used as a matrix for sustained release formulations. Hydrophobic polymers are suitably employed for preparing sustained release formulations, including pH-dependent and pH-independent polymers.

According to the present invention, to provide a sustained release tamsulosin HCl formulation, hydryphobic polymers (include pH-dependent and pH-independent polymers) may be employed to control the dissolution release profile of a tamsilosin formulation so that the formulation releases tamsulosin HCl slowly, stably and continuously as the formulation passed through the gastrointestinal tract.

The dissolution control capacity of such polymers is particularly important in a sustained release tamsulosin formulation because it may cause damping effects if the tamsulosin HCl is released too rapidly.

The hydrophobic polymers suitable for being employed according to the present invention are those capable of avoiding burst-out of drug during its residence in the gastrointestinal tract. Many materials have been known as the hydrophobic polymers in the pharmaceutical filed, include, but not limited to, methacrylic acid copolymer, sodium carboxymethyl cellulose, cellulose acetate, ethyl cellulose (EC), hydroxypropyl methyl-cellulose acetate succinate (HPMCAS) and cellulose acetate propionate (CAP). These polymers may be used alone or in combination in a sustained release tamsulosin formulation. The pH-dependent polymers are present in the tamsulosin formulation according to this invention in an amount ranging from about 10 to about 50% w/w of the formulation.

To prepare the sustained release tamsulosin formulation, water-soluble tamsulosin, a microsphere forming agent, a release-modulating agent and a diluent are mixed to obtain a mixture. Preferred microsphere forming agents may be employed for forming granules containing tamsulosin are glyceride or wax. Preferred release-modulating agents may be employed for delaying release rate of tamsulosin are the hydrophobic polymers. The mixture was added into a knead solution to form an oral dosage unit. An oral dosage unit of the sustained release tamsulosin formulation of this invention may be in the form of a capsule. The granules may be coated with one or more films for different purposes and then encapsulated.

In preferred embodiments of the present invention, the amount of tamsulosin HCl in the sustained release tamsulosin formulations is from about 0.03% to about 3% w/w of the formulation. The range of the concentration of the hydrophobic polymers in the sustained release tamsulosin formulations is about 10% to about 50% w/w of the formulation. The hydrophobic polymers may be employed to lower the fluctuation of the formulation in a human body. The range of the concentration of the microsphere forming agent in the sustained release tamsulosin formulations is about 10% to about 30% w/w of the formulation. The microsphere forming agent may also have the lubricating effect and make the procedure of granulation favorable. Also, the glue-like status of the acrylic polyol polymers under a high temperature can be improved by the use of a microsphere forming agent.

The sustained release tamsulosin formulation according to the present invention also contains a pharmaceutically acceptable diluent. The amount of the diluent in the sustained release tamsulosin formulation of the present invention is about 30% to about 50% w/w of the formulation.

More preferably, the amount of the concentration of the diluent in the sustained release tamsulosin formulation of the present invention is about 20% to about 40% w/w of the formulation.

The preferred diluent according to the present invention includes be lactose, starch, mannitol, sodium hydroxylpropyl cellulose, sodium starch, microcrystalline cellulose, glyceryl behenate, talcum powder, stearic acid, stearic salt or sodium stearyl fumarate, or a combination threreof.

The preferred microsphere forming agent according to the present invention includes glyceryl triacetate, glyceryl monostearate, glyceryl behenate, paraffin wax or carnauba wax, or a combination.

The techniques regarding film coating have become commonly available to a person skilled in the art. A method to make a film coating, for example, is described as follows. A film coating premix is dissolved in water and an organic solvent. The organic solvent used for preparing the film coating premix could be alcohol, acetone or isopropanol. Other diluents or anti-adhesive agents may be added into the above solvent mixture if necessary.

Other features and advantages of the present invention will be apparent from the following description of the preferred embodiments and from the claims.

### EXAMPLES

The following examples illustrate various aspects of the present invention but do not limit the claims in any manner whatsoever.

### Example 1

### Sustained Release Tamsulosin Formulation (1) and Method for the Production Thereof

Sustained release tamsulosin formulation (1)

| | | | |
|---|---|---|---|
| (a) | tamsulosin HCl | 4.90 | g |
| | co-processed polyvinyl acetate phthalate | 480 | g |
| | microcrystalline cellulose | 1080.0 | g |
| | glyceryl behenate | 631.1 | g |
| | ethylcellulose | 444 | g |
| (b) | film coat | | |
| | ethylcellulose | 150 | g |
| | triethyl citrate | 10.8 | g |

### Procedures:

The sustained release tamsulosin formulations according to the present invention are prepared as follows:

1. Tamsulosin HCl, co-processed polyvinyl acetate phthalate, microcrystalline cellulose, and glyceryl behenate are intimately mixed to obtain a mixture.

2. Ethylcellulose was wet-blended and then mixed with the mixture in an extruding granulator and centrifugal spheroider to form a granule.

3. The granules were dried in a tray dryer.

4. A film coat premix comprising ethylcellulose, and triethyl citrate were mixed well.

5. The dried granules were put into a fluidized bed coater, and the film coat premix dissolved in the selected solvent was sprayed on an outer surface of the granule.

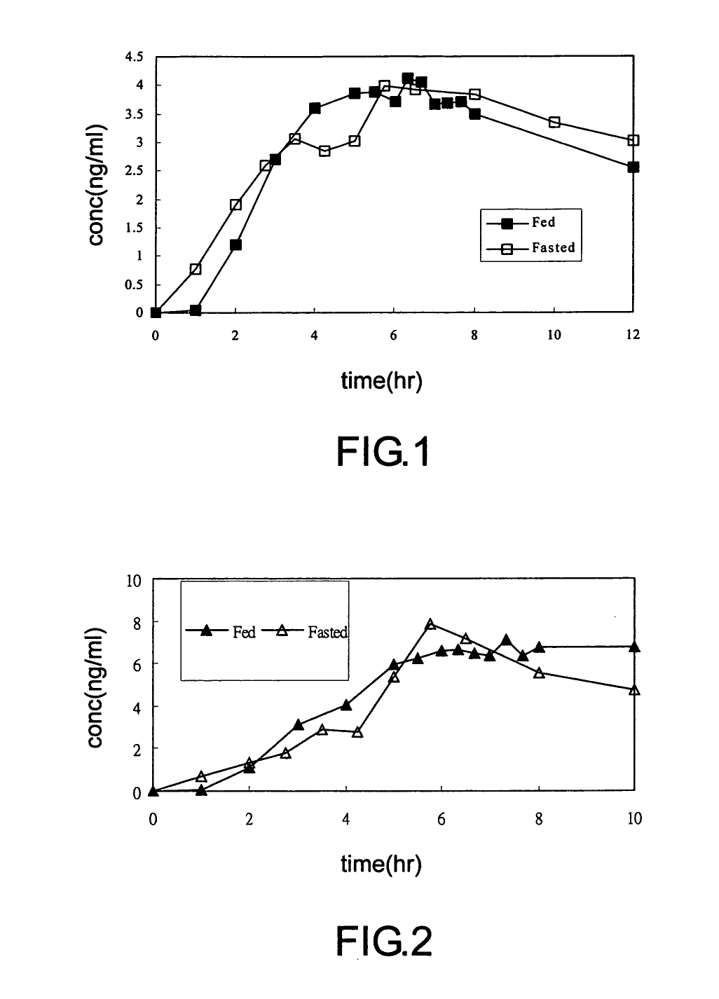

With reference to FIG. 1 and Tables 1 and 2, the capsules filled with granules obtained in Example 1 was orally administered to six adult male subjects under well-fed (fed) and empty-stomach (fasted) conditions, by a cross over method. Blood samples were withdrawn at definite time intervals and the concentration of tamsulosin HCl in plasma was measured by a validated analytical method. The data show that the sustained release tamsulosin formulation prepared according to the present invention releasing tamsulosin HCl very stable.

TABLE 1
The mean tamsulosin HCl plasma concentration of the
sustained release tamsulosin formulation obtained form
Example 1 after orally administering to six adult male subjects
under well-fed (fed) condition (with a standard meal).

| Time (hr) | Fed (ng/ml) |
|---|---|
| 0 | 0 |
| 1 | 0.05 |
| 2 | 1.20 |
| 3 | 2.70 |
| 4 | 3.60 |
| 5 | 3.87 |
| 5.5 | 3.88 |
| 6 | 3.71 |
| 6.33 | 4.11 |
| 6.67 | 4.06 |
| 7 | 3.67 |
| 7.33 | 3.68 |
| 7.67 | 3.72 |
| 8 | 3.49 |
| 12 | 2.55 |

TABLE 2
The mean tamsulosin HCl plasma concentration of the
sustained release tamsulosin formulation obtained form
Example 1 after orally administering to six adult male
subjects under empty-stomach (fasted) condition.

| Time (hr) | Fasted (ng/ml) |
|---|---|
| 0 | 0.00 |
| 1 | 0.77 |
| 2 | 1.90 |
| 2.75 | 2.59 |
| 3.5 | 3.06 |
| 4.25 | 2.86 |
| 5 | 3.03 |
| 5.75 | 3.98 |
| 6.5 | 3.92 |
| 8 | 3.84 |
| 10 | 3.35 |
| 12 | 3.02 |

### Example 2

### Sustained Release Tamsulosin Formulation (2) and Method for the Production Thereof

Sustained release tamsulosin formulation (2)

| | | | |
|---|---|---|---|
| (a) | tamsulosin HCl | 1.62 | g |
| | methacrylic acid copolymer | 160 | g |
| | microcrystalline cellulose | 360.0 | g |
| | triethyl citrate | 35.6 | g |
| | glyceryl behenate | 210.38 | g |
| | Ethylcellulose | 148.0 | g |
| (b) | film coat | | |
| | methacrylic acid copolymer | 57.12 | g |
| | 1N NH₃ | 19.29 | g |
| | talcum powder | 8.52 | g |
| | triethyl citrate | 28.58 | g |

### Procedures:

The sustained release tamsulosin formulations according to the present invention are prepared as follows:

1. Tamsulosin HCl, methacrylic acid copolymer, microcrystalline cellulose, and glyceryl behenate are intimately mixed to obtain a mixture.

2. Ethylcellulose and triethyl citrate was wet-blended and then mixed with the mixture in an extruding granulator and centrifugal spheroider to form a granule.

3. The granules were dried in a tray dryer.

4. A film coat premix comprising methacrylic acid copolymer, talcum powder and triethyl citrate were mixed well.

5. The dried granules were put into a fluidized bed coater, and the film coat premix dissolved in the selected solvent was sprayed on an outer surface of the granule.

With reference to FIG. 2 and Tables 3 and 4, the capsules filled with granules obtained in Example 2 was orally administered to six adult male subjects under well-fed (fed) and empty-stomach (fasted) conditions, by a cross over method. Blood samples were withdrawn at definite time intervals and the concentration of tamsulosin HCl in plasma was measured by a validated analytical method. The data show that the sustained release tamsulosin formulation prepared according to the present invention releasing tamsulosin HCl very stable.

TABLE 3
The mean tamsulosin HCl plasma concentration of the
sustained release tamsulosin HCl formulation obtained
form Example 2 after orally administering to six adult male
subjects under well-fed (fed) condition (with a standard meal).

| Time (hr) | Fed (ng/ml) |
|---|---|
| 0 | 0 |
| 1 | 0.06 |
| 2 | 1.10 |
| 3 | 3.11 |
| 4 | 4.04 |
| 5 | 5.96 |
| 5.5 | 6.23 |
| 6 | 6.62 |
| 6.33 | 6.62 |
| 6.67 | 6.46 |
| 7 | 6.38 |
| 7.33 | 7.10 |
| 7.67 | 6.33 |
| 8 | 6.79 |
| 10 | 6.75 |

TABLE 4
The mean tamsulosin HCl plasma concentration of the
sustained release tamsulosin HCl formulation obtained
form Example 2 after orally administering to six adult male
subjects under empty-stomach (fasted) condition.

| Time (hr) | Fasted (ng/ml) |
|---|---|
| 0 | 0.00 |
| 1 | 0.68 |
| 2 | 1.36 |
| 2.75 | 1.78 |
| 3.5 | 2.90 |
| 4.25 | 2.80 |
| 5 | 5.40 |
| 5.75 | 7.87 |
| 6.5 | 7.14 |
| 8 | 5.55 |
| 10 | 4.76 |

### Example 3

### Ingredient-Releasing Rate Test of a Commercial Sustained Release Capsules of the Prior Art

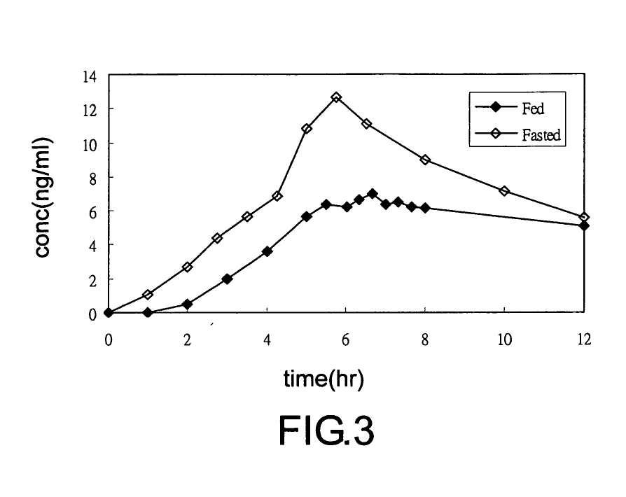

With reference to FIG. 3 and Tables 5 and 6, the commercial sustained release capsules (commercial name is Flomax (Boehringer Ingelheim) in the U.S.,) of the prior art was orally administered to twelve adult male subjects under well-fed (fed) and empty-stomach (fasted) conditions, by a cross over method. Blood samples were withdrawn at definite time intervals and the concentration of tamsulosin HCl in plasma was measured by a validated analytical method. The data show that the commercial sustained release tamsulosin formulation showing two different releasing rates under fed and fasted conditions. Therefore, the releasing rate of the commercial sustained release tamsulosin formulation is unstable.

TABLE 5
The mean tamsulosin HCl plasma concentration of the
commercial sustained release capsules of the prior art
after orally administering to twelve adult male subjects
under fed condition (with a standard meal).

| Time (hr) | Fed (ng/ml) |
|---|---|
| 0 | 0 |
| 1 | 0.01 |
| 2 | 0.51 |
| 3 | 1.97 |
| 4 | 3.57 |
| 5 | 5.63 |
| 5.5 | 6.39 |
| 6 | 6.24 |
| 6.33 | 6.62 |
| 6.67 | 6.98 |
| 7 | 6.39 |
| 7.33 | 7.48 |
| 7.67 | 6.20 |
| 8 | 6.15 |
| 12 | 5.07 |

TABLE 6
The mean tamsulosin HCl plasma concentration of the
commercial sustained release capsules of the prior art
after orally administering to twelve adult male
subjects under fasted condition.

| Time (hr) | Fasted (ng/ml) |
|---|---|
| 0 | 0.00 |
| 1 | 1.09 |
| 2 | 2.69 |
| 2.75 | 4.42 |
| 3.5 | 5.69 |
| 4.25 | 6.85 |
| 5 | 10.85 |
| 5.75 | 12.63 |
| 6.5 | 11.12 |
| 8 | 8.95 |
| 10 | 7.11 |
| 12 | 5.60 |

According to FIG. 1, FIG. 2 and FIG. 3, it is clear that feeding did not affect the pharmacokinetics of the sustained release tamsulosin formulations obtained in Example 1 and Example 2 of the present invention. On the contrast, the commercial sustained release tamsulosin capsules of the prior art showed the tamsulosin HCl plasma concentration was affected seriously by the food ingestion.

Accordingly, the foregoing examples illustrated the following advantages of the sustained release tamsulosin formulations of the present invention:

1. In the process present invention, the use of a microsphere forming agent as a lubricant was surprisingly found to solve the problem resulted from the glue-like status of an acrylic acid polymer or acrylic acid copolymer kneaded under a high temperature.

2. Sustained release tamsulosin formulations according to the present invention was found surprisingly to exhibit lack of a food effect. Therefore, patients can benefit from without remembering dosage recommendation (before or after feeding) of taking such formulations which may also result in an improved safety.

Various modifications and variations of the present invention will be recognized by those persons skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

What is claimed is:

1. A sustained release tamsulosin formulation, comprising
tamsulosin HCl or a pharmaceutically acceptable salt thereof,
a hydrophobic polymer present at about 10% to about 50% w/w of the formulation,
a microsphere forming agent present at about 10% to about 30% w/w of the formulation, and
a diluent present at about 30% to about 50% w/w of the formulation, wherein formulation releases less than 5% of tamsulosin HCl or the pharmaceutically acceptable salt thereof during the first two hours.

2. The sustained release tamsulosin formulation as claimed in claim 1, wherein the diluent is selected from the group consisting of lactose, starch, mannitol, sodium hydroxylpropyl cellulose, sodium starch, microcrystalline cellulose, glyceryl behenate, talcum powder, stearic acid, stearic salt and sodium stearyl fumarate.

3. The sustained release tamsulosin formulation as claimed in claim 1, wherein the hydrophobic polymer is pH-dependent polymers or pH-independent polymers.

4. The sustained release tamsulosin formulation as claimed in claim 3, wherein the hydrophobic polymer is selected from the group consisting of methacrylic acid copolymer, sodium carboxymethyl cellulose, cellulose acetate, ethyl cellulose (EC), hydroxypropyl methyl-cellulose acetate succinate (HPMCAS) and cellulose acetate phthalate (CAP).

5. The sustained release tamsulosin formulation as claimed in claim 1, wherein the microsphere forming agent is glyceryl triacetate, glyceryl monostearate, glyceryl behenate, paraffin wax or carnauba wax.

6. The sustained release tamsulosin formulation as claimed in claim 1, wherein tamsulosin HCl or the pharmaceutically acceptable salt thereof is present in the range of about 0.01% to about 3% w/w of the formulation.

7. The sustained release tamsulosin formulation as claimed in claim 6, wherein tamsulosin HCl or the pharmaceutically acceptable salt thereof is present in the range of about 0.03% to about 3% w/w of the formulation.

8. A method for preparing the sustained release tamsulosin formulation, comprising:
preparing a mixture by mixing a water-soluble tamsulosin HCl or the pharmaceutically acceptable salt thereof, a microsphere forming agent, a release modulating agent and a diluent,
preparing a film coating premix,
forming the mixture into granules, and
coating the film coating premix on the granules.

9. The method as claimed in claim 8, wherein the film coat premix is dissolved in a solvent selected from the group consisting of water and organic solvent.

10. The method as claimed in claim 8,wherein the hydrophobic polymer is selected from the group consisting of methacrylic acid copolymer, sodium carboxymethyl cellulose, cellulose acetate, ethyl cellulose (EC), hydroxypropyl methyl-cellulose acetate succinate (HPMCAS) and cellulose acetate phthalate (CAP).

11. The method as claimed in claim 8, wherein the microsphere forming agent is glyceryl triacetate, glyceryl monostearate, glyceryl behenate, paraffin wax or carnauba wax.

12. The method as claimed in claim 8, wherein the film coat premix is prepared using more than one of ethylcellulose, triethyl citrate, methacrylic acid copolymer and talcum powder.
